(19) United States
(12) Patent Application Publication
(10) Pub. No.: US 2025/0148355 A1
(43) Pub. Date: May 8, 2025

(54) SYSTEMS AND METHODS FOR INTELLIGENT RESOURCE BIDDING PLATFORM

(71) Applicant: Optum Services (Ireland) Limited, Dublin (IE)

(72) Inventors: James McNair SLOAN, Bathgate (GB); Alison Brigitte SANDERS, Hindlip (GB)

(21) Appl. No.: 18/503,436

(22) Filed: Nov. 7, 2023

(51) Int. Cl. G06N 20/00; G06F 9/50

(52) U.S. Cl. CPC G06N 20/00; G06F 9/5005

(57) ABSTRACT

A method performed by one or more processors of a computing system includes: receiving an entity attribute associated with an entity; generating an entity consideration dataset using the received entity attribute; providing the generated entity consideration dataset to a plurality of resource systems; receiving one or more responses from the plurality of resource systems in response to the provided entity consideration dataset; generating a resource consideration dataset using the received one or more responses; and matching a resource system of the plurality of resource systems with the entity based on the resource consideration dataset.

### TECHNICAL FIELD

The present disclosure generally relates to the field of data analytics and artificial intelligence. Particularly, the present disclosure relates to systems and methods for providing an intelligent bidding platform to match a resource system with an entity based on analyzing data sources indicative of various contributing factors.

### BACKGROUND

An entity may be in a condition to be discharged from an initial resource system, but may still not be in a condition to operate without any support from a subsequent resource system. The entity may thus require additional support from a subsequent resource system after being discharged from the initial resource system. The inability to quickly and efficiently locate an appropriate, post-discharge resource system for an entity results in the entity remaining in the initial resource system, which leads to a bottleneck in the initial resource system as well as related resource systems.

The present disclosure is directed to overcoming the above-mentioned challenges. The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY OF THE DISCLOSURE

In some aspects, the techniques described herein relate to a method performed by one or more processors of a computing system, the method including: receiving an entity attribute associated with an entity; generating an entity consideration dataset using the received entity attribute; providing the generated entity consideration dataset to a plurality of resource systems; receiving one or more responses from the plurality of resource systems in response to the provided entity consideration dataset; generating a resource consideration dataset using the received one or more responses; and matching a resource system of the plurality of resource systems with the entity based on the resource consideration dataset.

In some aspects, the techniques described herein relate to a method, wherein generating the entity consideration dataset using the received entity attribute includes: receiving metadata regarding the entity attribute; extracting a feature from the received metadata, the extracted feature corresponding to a feature of a trained machine-learning based model for determining the entity consideration dataset for the entity attribute based on a learned association between the extracted feature and a resource system; and automatically determining, using the trained machine-learning based model, the entity consideration dataset for the entity attribute based on the extracted feature and the learned association between the extracted feature and the resource system, wherein the trained machine-learning based model was trained based at least in part on a first feature extracted from metadata regarding a patient attribute and a second feature extracted from metadata regarding a resource system related to the entity attribute.

In some aspects, the techniques described herein relate to a method, wherein the trained machine-learning based model was trained by operations including: receiving first metadata regarding a first entity attribute; extracting a first feature from the received first metadata; receiving second metadata regarding a first resource system related to the first entity attribute; extracting a second feature from the received second metadata; training the machine-learning based model to learn an association between the first entity attribute and the first resource system related to the first entity attribute, based on the extracted first feature and the extracted second feature; and automatically determining, using the trained machine-learning based model, a first entity consideration set for the first entity attribute based on the extracted first feature and the learned association between the first entity attribute and the first resource system related to the first entity attribute.

In some aspects, the techniques described herein relate to a method, wherein generating the resource consideration dataset using the received one or more responses includes: receiving metadata regarding a resource system associated with the one or more responses; extracting a feature from the received metadata, the extracted feature corresponding to a feature of a trained machine-learning based model for determining the resource consideration dataset for the resource system based on a learned association between the extracted feature and an entity outcome; and automatically determining, using the trained machine-learning based model, the resource consideration dataset for the resource system based on the extracted feature and the learned association between the extracted feature and the entity outcome, wherein the trained machine-learning based model was trained based at least in part on a first feature extracted from metadata regarding a resource system and a second feature extracted from metadata regarding an entity outcome related to the resource system.

In some aspects, the techniques described herein relate to a method, wherein the trained machine-learning based model was trained by operations including: receiving first metadata regarding a first resource system; extracting a first feature from the received first metadata; receiving second metadata regarding a first entity outcome related to the first resource system; extracting a second feature from the received second metadata; training the machine-learning based model to learn an association between the first resource system and the first entity outcome related to the first resource system, based on the extracted first feature and the extracted second feature; and automatically determining, using the trained machine-learning based model, a first resource consideration dataset for the first resource system based on the extracted first feature and the learned association between the first resource system and the first entity outcome related to the first resource system.

In some aspects, the techniques described herein relate to a method, wherein matching the resource system with the entity based on the resource consideration dataset includes: receiving an acceptance of a response, among the one or more responses, from the resource system, the entity, a related entity, or an authorized resource system for the entity, and providing a notification to the resource system of the acceptance of the response.

In some aspects, the techniques described herein relate to a method, wherein generating the resource consideration dataset using the received one or more responses includes: generating a score for the resource consideration dataset; and generating a ranked list of resource consideration datasets, including the resource consideration dataset, based on the score for each of a plurality of resource consideration datasets.

In some aspects, the techniques described herein relate to a method, wherein generating the ranked list of resource consideration datasets includes using a trained machine-learning based model.

In some aspects, the techniques described herein relate to a method, wherein matching the resource system with the entity based on the resource consideration dataset includes: automatically accepting a response, among the one or more responses, from the resource system, among the plurality of resource systems, having a highest score in the ranked list of resource consideration datasets including the resource consideration dataset; and providing a notification to the resource system of the acceptance of the response.

In some aspects, the techniques described herein relate to a method, wherein the entity attribute includes one or more of: a characteristic of the entity, a condition of the entity, or a treatment of the entity.

In some aspects, the techniques described herein relate to a method, wherein the entity consideration dataset includes one or more of: a cost of providing a service to the entity, a likelihood of the entity needing additional services the resource system can provide, a likelihood of the entity needing additional services the resource system cannot provide, a probability of the entity having a condition that is not documented, or a demand and capacity planning of the resource system.

In some aspects, the techniques described herein relate to a method, wherein the resource consideration dataset includes factors including one or more of: a community fit for the entity, a causal inference model forecasting an additional life expectancy by a service provided for each response, or an ability for the entity to meet a cost of services over a given time period.

In some aspects, the techniques described herein relate to a method, wherein generating the resource consideration dataset using the received one or more responses includes: generating a score for the resource consideration dataset based on a weighting scheme for the factors; and generating a ranked list of resource consideration datasets, including the resource consideration dataset, based on the score for each of a plurality of resource consideration datasets.

In some aspects, the techniques described herein relate to a system including: one or more processors; and at least one memory storing instructions that, when executed by the one or more processors, cause the one or more processors to perform operations including: receiving an entity attribute associated with an entity; generating an entity consideration dataset using the received entity attribute; providing the generated entity consideration dataset to a plurality of resource systems; receiving one or more responses from the plurality of resource systems in response to the provided entity consideration dataset; generating a resource consideration dataset using the received one or more responses; and matching a resource system of the plurality of resource systems with the entity based on the resource consideration dataset.

In some aspects, the techniques described herein relate to a system, wherein one or more of generating the entity consideration dataset using the received entity attribute or generating the resource consideration dataset using the received one or more responses includes using a trained machine-learning based model.

In some aspects, the techniques described herein relate to a system, wherein the operations further include: receiving an acceptance of a response, among the one or more responses, from the resource system, the entity, a related entity, or an authorized resource system for the entity, and providing a notification to the resource system of the acceptance of the response.

In some aspects, the techniques described herein relate to a system, wherein generating the resource consideration dataset using the received one or more responses includes: generating a score for the resource consideration dataset; and generating a ranked list of resource consideration datasets, including the resource consideration dataset, based on the score for each of a plurality of resource consideration datasets.

In some aspects, the techniques described herein relate to a system, wherein generating the ranked list of resource consideration datasets includes using a trained machine-learning based model.

In some aspects, the techniques described herein relate to a system, wherein matching the resource system with the entity based on the resource consideration dataset includes: automatically accepting a response, among the one or more responses, from the resource system, among the plurality of resource systems, having a highest score in the ranked list of resource consideration datasets including the resource consideration dataset; and providing a notification to the resource system of the acceptance of the response.

In some aspects, the techniques described herein relate to a non-transitory computer readable medium storing instructions that, when executed by one or more processors, cause the one or more processors to perform operations including: receiving an entity attribute associated with an entity; generating an entity consideration dataset using the received entity attribute; providing the generated entity consideration dataset to a plurality of resource systems; receiving one or more responses from the plurality of resource systems in response to the provided entity consideration dataset; generating a resource consideration dataset using the received one or more responses; and matching a resource system of the plurality of resource systems with the entity based on the resource consideration dataset.

Additional objects and advantages of the disclosed embodiments will be set forth in part in the description that follows, and in part will be apparent from the description, or may be learned by practice of the disclosed embodiments. The objects and advantages of the disclosed embodiments will be realized and attained by means of the elements and combinations particularly pointed out in the appended claims.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 depicts an exemplary system infrastructure for a bidding platform, according to one or more embodiments.

FIG. 2 depicts an exemplary data model for a bidding platform, according to one or more embodiments.

FIG. 3 depicts a flowchart of a method of providing a bidding platform to match a health care provider with a patient, according to one or more embodiments.

FIG. 4 depicts a flowchart of a method of determining a patient consideration using a trained machine-learning based model, according to one or more embodiments.

FIG. 5 depicts a flowchart of a method of training a machine-learning based model to determine a patient consideration, according to one or more embodiments.

FIG. 6 depicts a flowchart of a method of determining a provider consideration using a trained machine-learning based model, according to one or more embodiments.

FIG. 7 depicts a flowchart of a method of training a machine-learning based model to determine a provider consideration, according to one or more embodiments.

FIG. 8 depicts an implementation of a computer system that executes techniques presented herein, according to one or more embodiments.

### DETAILED DESCRIPTION OF EMBODIMENTS

The present disclosure relates to systems and methods for providing an intelligent bidding platform to match a resource system with an entity based on analyzing data sources indicative of various contributing factors.

More particularly, and without limitation, various embodiments of the present disclosure are applicable in the healthcare industry, and examples of embodiments applied in the healthcare industry are provided herein. For example, the present disclosure discusses systems and methods for providing a bidding platform to match a health care provider with a patient, and, more particularly, to systems and methods for a bidding platform with patient considerations for providers and provider considerations for patients, to match a health care provider with a patient. However, as would be apparent to a person of ordinary skill, the embodiments of the present disclosure can be applicable in various other industries, where there is a need for an effective computing platform capable of connecting an entity, subject, or object that is processed or serviced at an earlier system to a subsequent system that is optimized for further processing or servicing of the entity, subject, or object.

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section.

In the United Kingdom, for example, the National Health Service (NHS) is collectively made up of over 100 companies which focus on separate elements of the healthcare system. Secondary care and community care, for example, are separate systems, and are guided by different incentives and payment structures. Secondary care in the UK includes hospital care, including elective, urgent, emergency, or mental health care. The NHS Long Term Plan focuses each individual organization towards common goals, one of which is integrated care, which encourages the many organizations to better collaborate to improve healthcare outcomes and lower costs.

A patient may be well enough to be discharged from secondary care, but may not be well enough to live unsupported, and thus may require community care services such as private residential care or supporting services. This results in the patient remaining in secondary care while nurses try to arrange services, which leads to a bottleneck in the healthcare system.

For example, patients who are ready to be discharged, but remain in secondary care, delay patients waiting to receive treatment. Hospitals missing discharge targets between January and March 2019 could have undertaken 17,715 more operations. Additionally, secondary healthcare is expensive, which adds additional cost to the patient's stay. Patients who are ready to be discharged but remain in secondary care delay patients in an accident and emergency department when they cannot be sent to the appropriate department. Elderly patients experience a significant functional decline during hospital stays (e.g. 35% of 70 year old patients, and 65% of 90 year old patients, compared to baselines). Organizing discharges to the appropriate service are a significant pressure on frontline staff, adding to already significant pressures and stresses. Therefore, the timely discharge of patients is crucial to allow efficient healthcare and reduce the burden on patients and the NHS.

Some solutions involve nurses spending intensive time liaising with local services and families to find appropriate local care services to meet the patient's needs. However, these solutions are very time-intensive and labor-intensive, due to nurses calling different services and residential care providers that meet the patient's needs. The nurse's time could be better spent on delivering care for patients and other duties, and reducing pressure and stress on staff to rapidly organize discharge with incoming patients.

Some solutions do not have a bidding mechanism, do not have a two-sided platform, and provide services only at contracted prices. This can lead to inefficiencies in pricing, and reduces patient (i.e. consumer) choice. Also, some solutions require significant nurse input to select and discharge patients. One or more embodiments include a bidding mechanism. One or more embodiments include a two-sided platform. One or more embodiments include making patient data available to healthcare service providers.

Some solutions offer a marketplace and platform where patients can connect with medical professionals to select the best bid. Some solutions allow the medical providers to create a profile and include relevant information such as training, specialties, quality outcomes, and reviews. The patient reviews and selects the best bid. Some solutions present information to the patient, but do not make personalized recommendations for the patient using machine learning. Some solutions only allow a patient to select their facility. One or more embodiments provide personalized recommendations for the patient using machine learning. One or more embodiments include a bidding platform that allows families or nurses or authorized third parties to select a personalized best bid if a patient is unable to select a bid.

Some solutions offer information and advice to patients, or families of patients, looking for a care home. However, these solutions are only a research tool, and do not make any personalized recommendations for the patient. Some solutions compare costs and services from professional caregivers. However, these solutions are only a comparison tool, and do not make personalized recommendations for the patient.

One or more embodiments include an automated two-sided computing platform where waiting-to-be-discharged patients (e.g., entities) and private healthcare service providers (e.g., resource systems) can be matched, which allows a quicker discharge of patients, and frees healthcare providers from this task. For example, when a patient is ready to be discharged, healthcare staff initiate a process which provides anonymized patient data to a platform for bids from healthcare service providers. One or more embodiments include machine learning models on the platform to allow both sides of the platform (patient and service providers) to make informed decisions.

One or more embodiments include machine learning models that estimate and forecast a range of data points to consider when making a bid for a patient. Given patient data and service provider data, the machine learning models provide estimates to the service provider that are relevant to the bid the service providers make for a patient. The estimates to the service provider include one or more of a bid the service provider should make for a patient, a cost of providing service to the given patient, a likelihood of a patient needing additional services the service provider can provide, a likelihood of a patient needing additional services the service provider cannot provide, probabilities of patient having a condition that is not documented, or demand and capacity planning of the service provider. One or more embodiments include a machine learning model that forecasts total patient treatment cost so that healthcare service providers have an estimate of the expense of providing a service to the given patient, thereby providing a bid price to achieve a target profitability margin.

One or more embodiments receive a bid from service providers to meet needs of a patient, given the one or more of the above estimates. One or more embodiments present the bids to the patient, alongside a range of data points and considerations (e.g., entity consideration datasets) from the machine learning models. Given a range of bids from service providers, one or more embodiments use machine learning to provide a range of data points for the patient to consider (e.g., resource consideration datasets). The estimates to the patient include one or more of a community fit for the patient, a causal inference model forecasting the additional life expectancy by the service provided for each bid, or an ability for a patient to meet the costs of the services over a given time period. The community fit for the patient eases a patient's integration into a service so that the patient is at ease. The causal inference model forecasts additional life expectancy if a patient is sent to a specific provider, as some service providers provide a superior service which results in better health outcomes for the given patient.

Given a range of bids from service providers, one or more embodiments use machine learning to provide a personalized ranked list of bids that is most likely to align with needs and wants of a patient. One or more embodiments provide an interface for a patient to select a weighting scheme across factors including on or more of cost, community fit, or additional life expectancy, for example. One or more embodiments generate a personalized list of bids using the selected weighting scheme. One or more embodiments receive an acceptance of a bid, among the personalized list of bids, from a patient that is most suited to their needs and wants, and the patient is discharged appropriately. One or more embodiments automatically accept a bid, among the personalized list of bids, of a service provider with a highest score in the personalized list of bids, and the patient is discharged appropriately.

One or more embodiments represent a patient's data (as a patient) by a complete and anonymized medical history, and include current conditions and required daily treatments. Given this information, appropriate healthcare service providers (bidders) submit informed bids to treat that patient. The patient and/or family then decide which bidder to select, and a nurse discharges the patient to the successful bidder.

One or more embodiments provide multiple benefits to nurses, patients, healthcare service providers, and the wider healthcare system. One or more embodiments reduce costs, as patients are discharged in a timely manner, by reducing time and cost of patients who are ready to be discharged but remain in secondary care, which is more expensive than more appropriate care in a community setting, for example. One or more embodiments reduce nurse workloads, as a nurse no longer manually telephones multiple service providers to find suitable services with capacity to treat the patient. One or more embodiments increase an efficiency of services by introducing a bidding mechanism, which reduces costs to patients due to increased competition among service providers. One or more embodiments reduce the delay in a patient being discharged. One or more embodiments decrease the bottleneck or resource-intensiveness that may be present in the computing system(s) of the healthcare provider(s) due to a delay in locating an appropriate subsequent healthcare provider. One or more embodiments encourage more collaboration between secondary care and community care. One or more embodiments automatically provide a personalized list of bids, which frees nurses for other caring tasks.

Some solutions lead to inefficiently priced and expensive services, as some service providers are undeservedly preferred. One or more embodiments provide a platform to facilitate matching between patients and service providers, which reduces a burden on nurses and frees them for other healthcare tasks. One or more embodiments provide a bidding mechanism, which increases competition among healthcare service providers, reduces patient cost, and increases patient satisfaction. One or more embodiments provide machine learning models to help both sides (patients and bidders) of the platform make more informed decisions, which lowers patient costs, and allow healthcare service providers to charge patients appropriately and not receive patients with unexpected needs. One or more embodiments provide machine learning models that analyze various types of data associated with patients and healthcare service providers, which lead to a more accurate determination of healthcare service providers for a patient that is optimized for accommodating the patient's needs and circumstances.

One or more embodiments provide one or more of (i) machine learning based estimates for service providers to make more informed bids on a patient, (ii) machine learning based estimates for patients to consider bids, or (iii) machine learning based personalized ranked list of bids for patients. With these models, patients have additional data points to make informed decisions, in addition to the offered price per bid from healthcare service providers. For example, a model forecasts a patient's life expectancy for each bidding healthcare provider; and the patient then considers the cost of the care and an estimate of quality of care for each bidder.

In one or more embodiments, healthcare service providers can only bid on the platform if the providers have the appropriate qualifications, licenses, certification, and legal permissions to provide the service required for the patient, and can only bid for patients where they legally able to provide that specific service. For example, one or more embodiments prevent a healthcare provider that is only licensed to provide ablution-at-home services from submitting a bid to administer a patient's medication at home.

One or more embodiments capture data to improve cooperation between secondary care and community care. One or more embodiments provide a bidding platform for a service provider to match with a single patient, or to match with a cohort of patients with similar conditions and treatment needs.

One of the machine learning techniques that is useful and effective for the platform is a neural network, which is a type of supervised machine learning. Nonetheless, it should be noted that other machine learning techniques and frameworks may be used to perform the methods contemplated by the present disclosure. For example, the systems and methods may be realized using other types of supervised machine learning such as regression problems, random forest, etc., using unsupervised machine learning such as cluster algorithms, principal component analysis (PCA), etc., and/or using reinforcement learning.

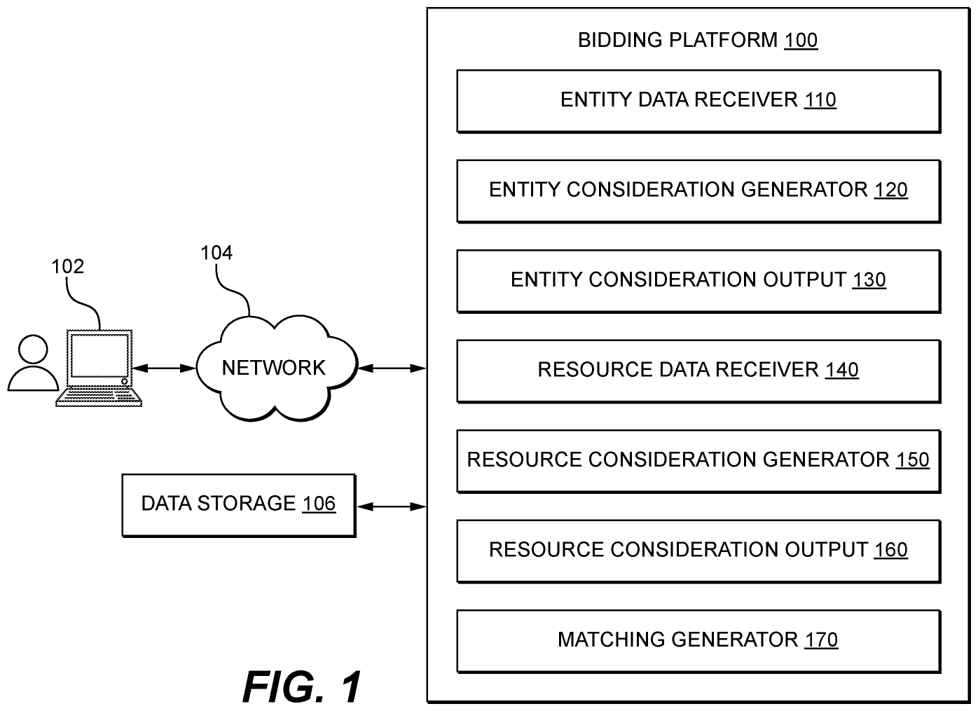

FIG. 1 depicts an exemplary system infrastructure for a bidding platform, according to one or more embodiments. As shown in FIG. 1, client device 102 communicates with bidding platform 100 over network 104. Data storage 106 communicates directly with bidding platform 100 as shown in FIG. 1, or communicates with bidding platform 100 over network 104. Bidding platform 100 includes entity data receiver 110, entity consideration generator 120, entity consideration output 130, resource data receiver 140, resource consideration generator 150, resource consideration output 160, and matching generator 170. Bidding platform 100 provides one or more of (i) machine learning based estimates for service providers to make more informed bids on a patient, (ii) machine learning based estimates for patients to consider bids, or (iii) machine learning based personalized ranked list of bids for patients.

Bidding platform 100 provide a bidding mechanism and a two-sided platform for making patient data available to healthcare service providers. Bidding platform 100 provide a bidding platform that allows families or nurses or authorized third parties to select a personalized best bid if a patient is unable to select a bid. Bidding platform 100 provide personalized recommendations for the patient. Bidding platform 100 provide a two-sided platform where waiting-to-be-discharged patients and private healthcare service providers can be matched, which allows a quicker discharge of patients, and frees healthcare providers from this task. Bidding platform 100 includes machine learning models on the platform to allow both sides of the platform (patient and service providers) to make informed decisions. Bidding platform 100 includes machine learning models that estimate and forecast a range of data points for a provider to consider when making a bid for a patient.

Bidding platform 100 receives a bid (e.g., a response) from healthcare service providers (e.g., resource systems) to meet needs of a patient (e.g., an entity), given the one or more of the above estimates. Bidding platform 100 presents the bids to the patient, alongside a range of data points and considerations (e.g., a resource consideration dataset) from the machine learning models. Given a range of bids from service providers, bidding platform 100 uses machine learning to provide a range of data points for the patient to consider.

Given a range of bids from service providers, bidding platform 100 uses machine learning to provide a personalized ranked list of bids that is most likely to align with needs and wants of a patient. Bidding platform 100 provide an interface for a patient to select a weighting scheme across factors including on or more of cost, community fit, or additional life expectancy, for example. Bidding platform 100 generates a personalized list of bids using the selected weighting scheme. Bidding platform 100 receives an acceptance of a bid, among the personalized list of bids, from a patient that is most suited to their needs and wants, and the patient is discharged appropriately. Bidding platform 100 automatically accepts a bid, among the personalized list of bids, of a service provider with a highest score in the personalized list of bids, and the patient is discharged appropriately.

Entity data receiver 110 receives patient data (e.g., entity data), such as patient attributes (e.g., entity attributes), through an interface of bidding platform 100 or through a data upload to bidding platform 100, for example. For example, when a patient is ready to be discharged, healthcare staff initiates a process which provides anonymized patient data to bidding platform 100 for bids from healthcare service providers. Entity data receiver 110 receives a patient's data (as a patient) by a complete and anonymized medical history, and includes current conditions and required daily treatments.

Entity consideration generator 120 includes machine learning models that estimate and forecast a range of data points for a provider to consider (e.g., an entity consideration dataset) when making a bid for a patient. Given patient data and service provider data, the machine learning models provide estimates to the service provider that are relevant to the bid the service provider makes for a patient. The estimates to the service provider includes one or more of a bid the service provider should make for a patient, a cost of providing service to the given patient, a likelihood of a patient needing additional services the service provider can provide, a likelihood of a patient needing additional services the service provider cannot provide, probabilities of patient having a condition that is not documented, or demand and capacity planning of the service provider. For example, entity consideration generator 120 includes a machine learning model that forecasts total patient treatment cost so that healthcare service providers have an estimate of the expense of providing a service to the given patient, thereby providing a bid price to achieve a target profitability margin.

Entity consideration output 130 provides the patient consideration from entity consideration generator 120 through an interface (e.g. a display) of bidding platform 100 or through a data transmission from bidding platform 100, for example. Resource data receiver 140 receives a bid from service providers to meet needs of a patient, given the one or more of the above estimates.

Resource data receiver 140 receives a bid (e.g., a response) from service providers through an interface of bidding platform 100 or through a data upload to bidding platform 100, for example. The bid from a service provider includes one or more of a cost to the patient, an availability date of the service, historic performance and quality of care provided, distance from a community of the patient, or demographics of current clients of the provider. This information is updated for each bid and/or is updated on a periodic basis through resource data receiver 140.

Resource consideration generator 150 is given a range of bids from service providers, and uses machine learning to provide a range of data points for the patient to consider (e.g., a resource consideration dataset). The estimates to the patient includes one or more of a community fit for the patient, a causal inference model forecasting the additional life expectancy by the service provided for each bid, or an ability for a patient to meet the costs of the services over a given time period. The community fit for the patient eases a patient's integration into a service so that the patient is at ease. The causal inference model forecasts additional life expectancy if a patient is sent to a specific provider, as some service providers provide a superior service which results in better health outcomes for the given patient.

Given a range of bids from service providers, resource consideration generator 150 uses machine learning to provide a personalized ranked list of bids that is most likely to align with needs and wants of a patient. Resource consideration generator 150 provide an interface for a patient to select a weighting scheme across factors including on or more of cost, community fit, or additional life expectancy, for example. Resource consideration generator 150 generates a personalized list of bids using the selected weighting scheme. Resource consideration output 160 presents the bids to the patient, alongside a range of data points and considerations from the machine learning models.

Matching generator 170 receives a selection of a bid from the patient and/or family, and arranges a discharge of the patient to the successful bidder. Matching generator 170 receives an acceptance of a bid, among the personalized list of bids, from a patient that is most suited to their needs and wants, and the patient is discharged appropriately. Matching generator 170 automatically accepts a bid, among the personalized list of bids, of a service provider with a highest score in the personalized list of bids, and the patient is discharged appropriately.

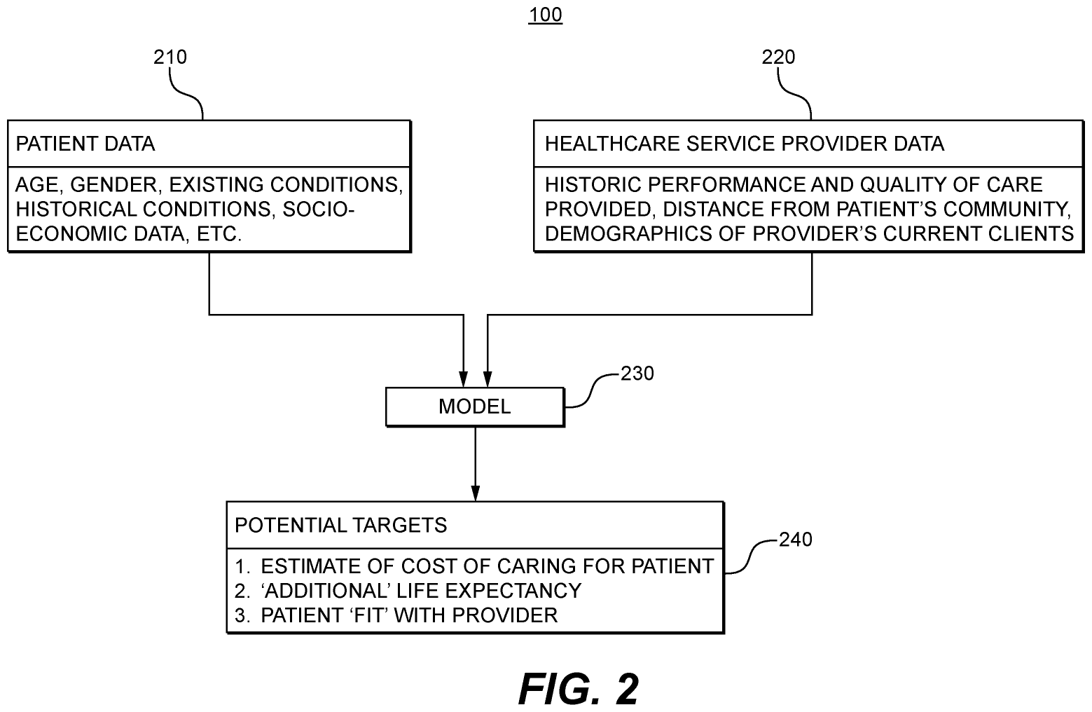

FIG. 2 depicts an exemplary data model for a bidding platform, according to one or more embodiments. As shown in FIG. 2, bidding platform 100 receives patient data 210 (e.g., entity data 210) and provider data 220 (e.g., resource data 220), and generates consideration data 240 using model 230.

Patient data 210 includes a patient attribute (e.g., an entity attribute) associated with the patient. The patient attribute includes one or more of a characteristic of the patient, a health condition of the patient, or a health treatment of the patient. A characteristic of the patient includes information such as gender and age, for example (e.g. male, 85 years old). A health condition of the patient includes information such as dementia, high blood pressure, diabetes, or poor mobility, for example. A health treatment of the patient includes information such as insulin treatment or complex pill schedule, for example.

Provider data 220 includes one or more of a cost to the patient, an availability date of the service, historic performance and quality of care provided, distance from a community of the patient, or demographics of current clients of the provider. This information is updated for each bid and/or is updated on a periodic basis.

Consideration data 240 includes one or more of a cost of the services over a given time period, additional life expectancy by the service, or patient fit with the provider.

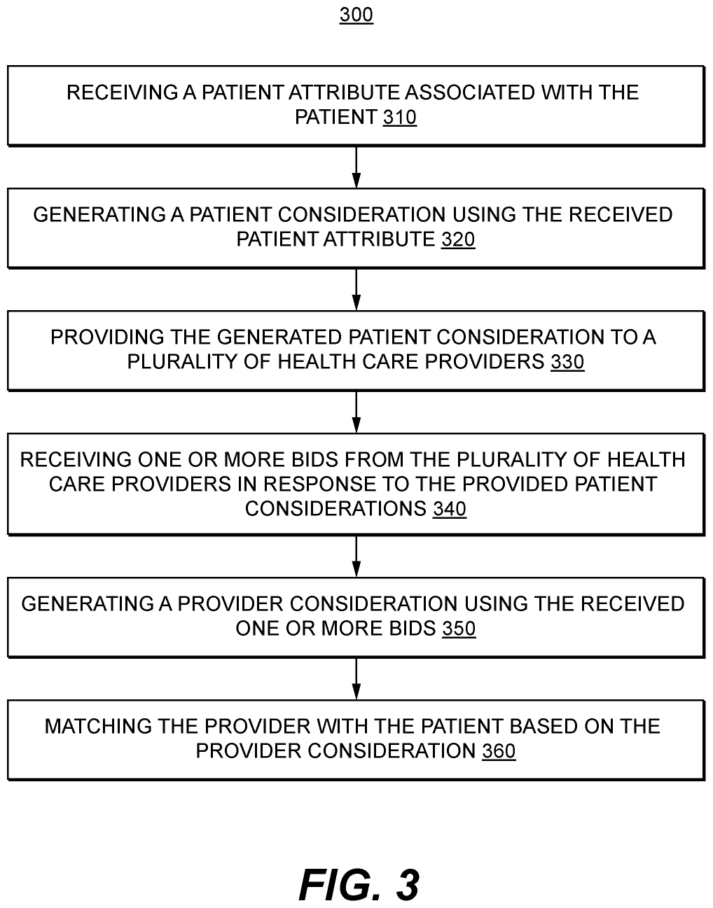

FIG. 3 depicts a flowchart of a method 300 performed by a bidding platform to match a health care provider (e.g., a resource system) with a patient (e.g., an entity), according to one or more embodiments. Operation 310 includes receiving a patient attribute (e.g., an entity attribute) associated with the patient. The patient attribute includes one or more of a characteristic of the patient, a health condition of the patient, or a health treatment of the patient. A characteristic of the patient includes information such as gender and age, for example (e.g. male, 85 years old). A health condition of the patient includes information such as dementia, high blood pressure, diabetes, or poor mobility, for example. A health treatment of the patient includes information such as insulin treatment or complex pill schedule, for example.

Operation 320 includes generating a patient consideration (e.g., an entity consideration dataset) using the received patient attribute. The patient consideration includes one or more of a cost of providing a health care service to the patient, a likelihood of the patient needing additional services the provider can provide, a likelihood of the patient needing additional services the provider cannot provide, a probability of the patient having a condition that is not documented, or a demand and capacity planning of the provider, for example.

Operation 330 includes providing the generated patient consideration to a plurality of health care providers. Operation 340 includes receiving one or more bids (e.g., responses) from the plurality of health care providers in response to the provided patient considerations. Operation 350 includes generating a provider consideration (e.g., a resource consideration database) using the received one or more bids. The provider consideration includes factors including one or more of a community fit for the patient, a causal inference model forecasting an additional life expectancy by a service provided for each bid, or an ability for the patient to meet a cost of services over a given time period, for example. Operation 360 includes matching the provider with the patient based on the provider consideration.

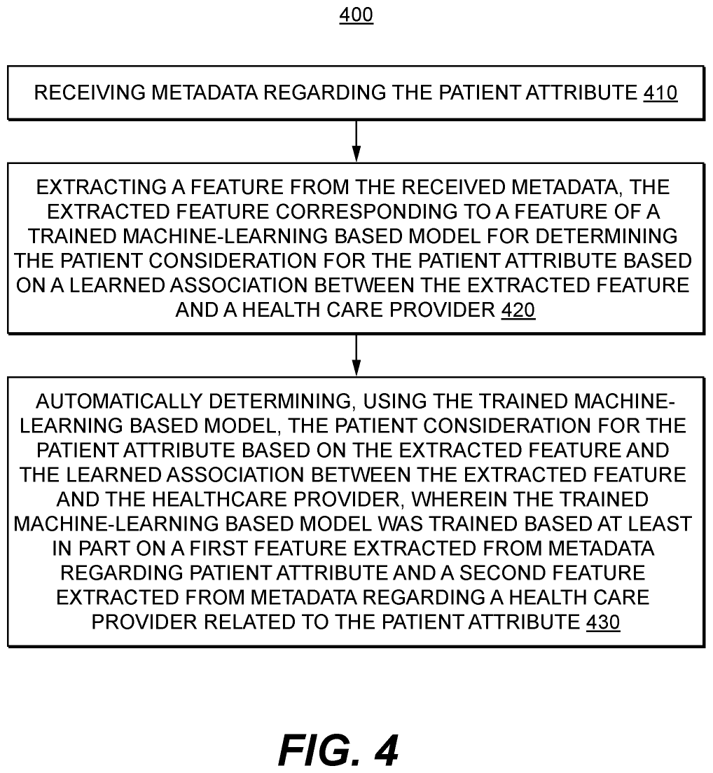

FIG. 4 depicts a flowchart of a method 400 performed by a bidding platform to determine a patient consideration (e.g., an entity consideration dataset) using a trained machine-learning based model, according to one or more embodiments. Operation 410 includes receiving metadata regarding the patient attribute. For example, a patient attribute includes one or more of a characteristic of the patient, a health condition of the patient, or a health treatment of the patient. A characteristic of the patient includes information such as gender and age, for example (e.g. male, 85 years old). A health condition of the patient includes information such as dementia, high blood pressure, diabetes, or poor mobility, for example. A health treatment of the patient includes information such as insulin treatment or complex pill schedule, for example.

Operation 420 includes extracting a feature from the received metadata, the extracted feature corresponding to a feature of a trained machine-learning based model for determining the patient consideration for the patient attribute based on a learned association between the extracted feature and a health care provider. Operation 430 includes automatically determining, using the trained machine-learning based model, the patient consideration for the patient attribute based on the extracted feature and the learned association between the extracted feature and the healthcare provider, wherein the trained machine-learning based model was trained based at least in part on a first feature extracted from metadata regarding a patient attribute and a second feature extracted from metadata regarding a health care provider related to the patient attribute.

The patient consideration includes one or more of a cost of providing a health care service to the patient, a likelihood of the patient needing additional services the provider can provide, a likelihood of the patient needing additional services the provider cannot provide, a probability of the patient having a condition that is not documented, or a demand and capacity planning of the provider, for example.

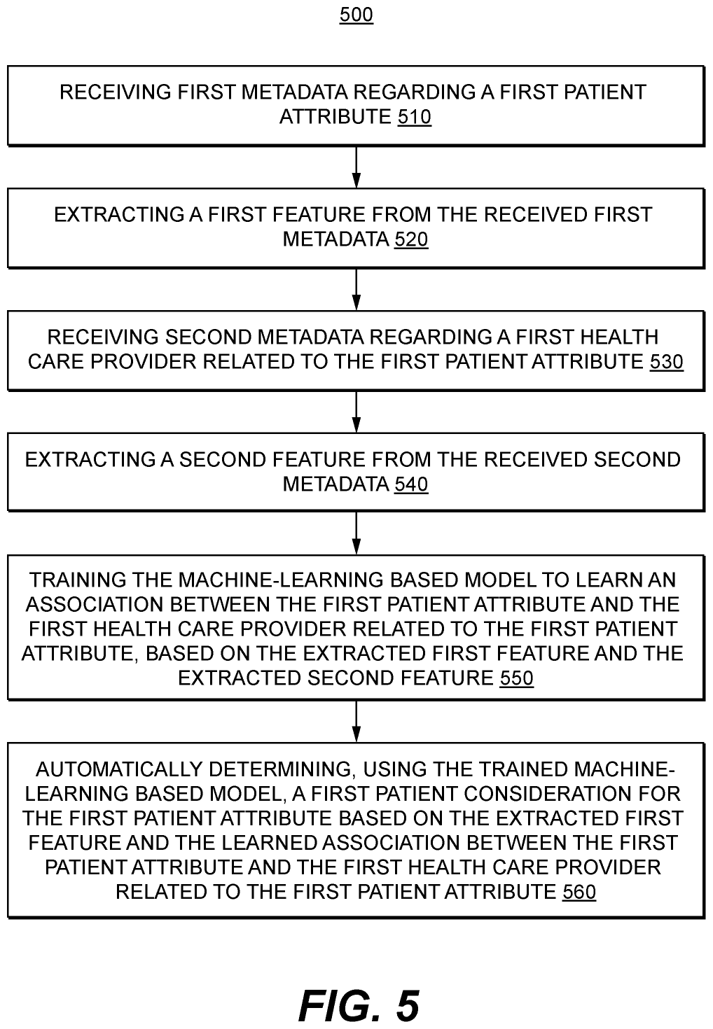

FIG. 5 depicts a flowchart of a method 500 performed by a bidding platform, or another computing device, to train a machine-learning based model to determine a patient consideration, according to one or more embodiments. Operation 510 includes receiving first metadata regarding a first patient attribute. Operation 520 includes extracting a first feature from the received first metadata. Operation 530 includes receiving second metadata regarding a first health care provider related to the first patient attribute. Operation 540 includes extracting a second feature from the received second metadata. Operation 550 includes training the machine-learning based model to learn an association between the first patient attribute and the first health care provider related to the first patient attribute, based on the extracted first feature and the extracted second feature. Operation 560 includes automatically determining, using the trained machine-learning based model, a first patient consideration for the first patient attribute based on the extracted first feature and the learned association between the first patient attribute and the first health care provider related to the first patient attribute.

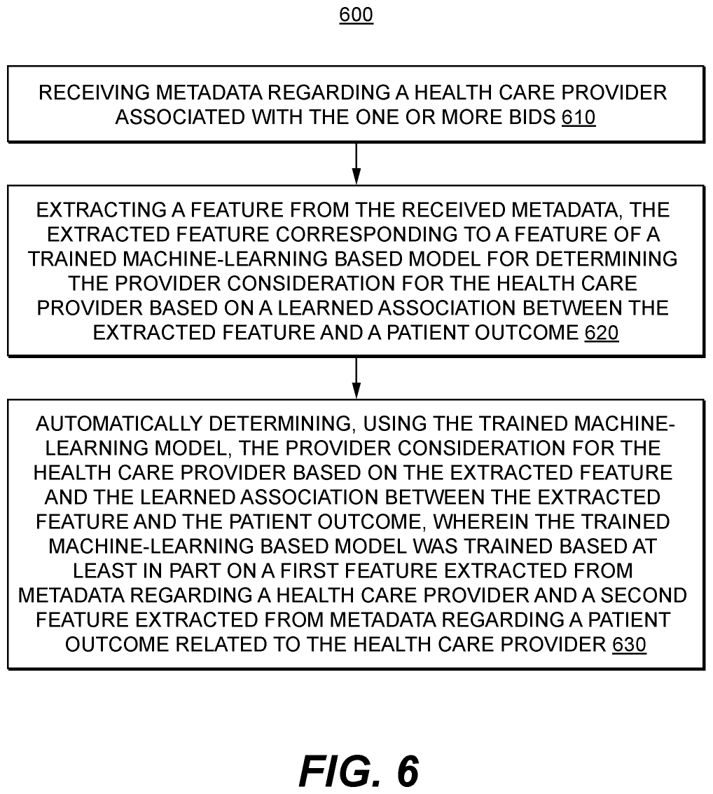

FIG. 6 depicts a flowchart of a method 600 performed by a bidding platform to determine a provider consideration using a trained machine-learning based model, according to one or more embodiments. Operation 610 includes receiving metadata regarding a health care provider associated with the one or more bids. The bid from a health care provider includes one or more of a cost to the patient, an availability date of the service, historic performance and quality of care provided, distance from a community of the patient, or demographics of current clients of the provider. This information is updated for each bid and/or is updated on a periodic basis.

Operation 620 includes extracting a feature from the received metadata, the extracted feature corresponding to a feature of a trained machine-learning based model for determining the provider consideration for the health care provider based on a learned association between the extracted feature and a patient outcome (e.g., an entity outcome). Operation 630 includes automatically determining, using the trained machine-learning based model, the provider consideration for the health care provider based on the extracted feature and the learned association between the extracted feature and the patient outcome, wherein the trained machine-learning based model was trained based at least in part on a first feature extracted from metadata regarding a health care provider and a second feature extracted from metadata regarding a patient outcome related to the health care provider.

The provider consideration includes one or more of a community fit for the patient, a causal inference model forecasting the additional life expectancy by the service provided for each bid, or an ability for a patient to meet the costs of the services over a given time period. The community fit for the patient eases a patient's integration into a service so that the patient is at ease. The causal inference model forecasts additional life expectancy if a patient is sent to a specific provider, as some service providers provide a superior service which results in better health outcomes for the given patient.

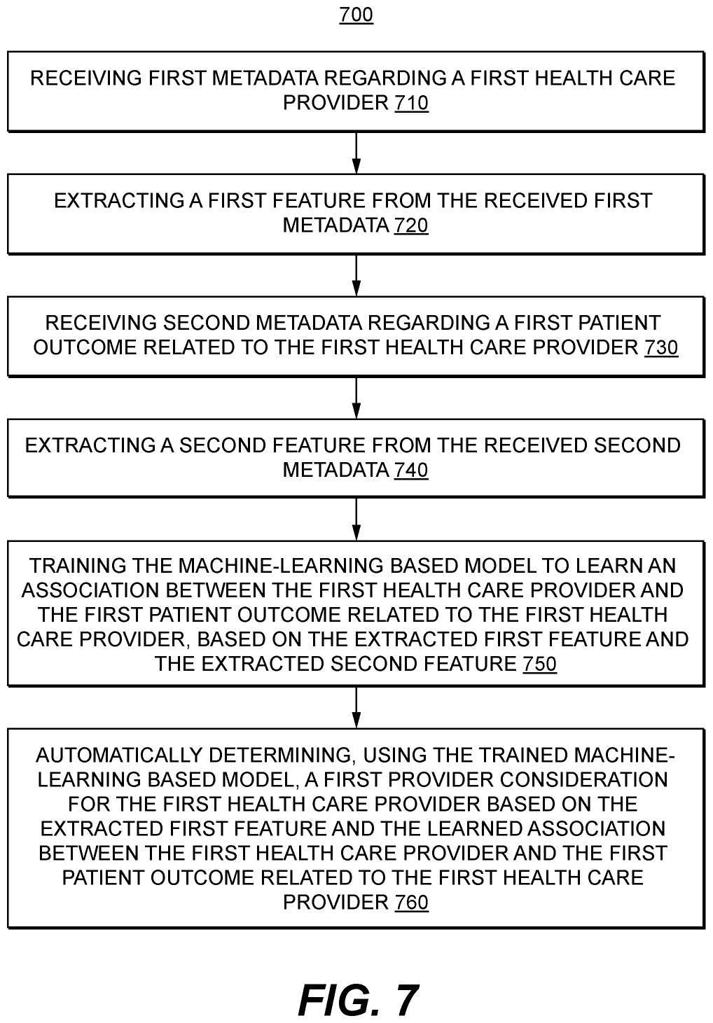

FIG. 7 depicts a flowchart of a method 700 performed by a bidding platform, or another computing device, to train a machine-learning based model to determine a provider consideration, according to one or more embodiments. Operation 710 includes receiving first metadata regarding a first health care provider. Operation 720 includes extracting a first feature from the received first metadata. Operation 730 includes receiving second metadata regarding a first patient outcome related to the first health care provider. Operation 740 includes extracting a second feature from the received second metadata. Operation 750 includes training the machine-learning based model to learn an association between the first health care provider and the first patient outcome related to the first health care provider, based on the extracted first feature and the extracted second feature. Operation 760 includes automatically determining, using the trained machine-learning based model, a first provider consideration for the first health care provider based on the extracted first feature and the learned association between the first health care provider and the first patient outcome related to the first health care provider.

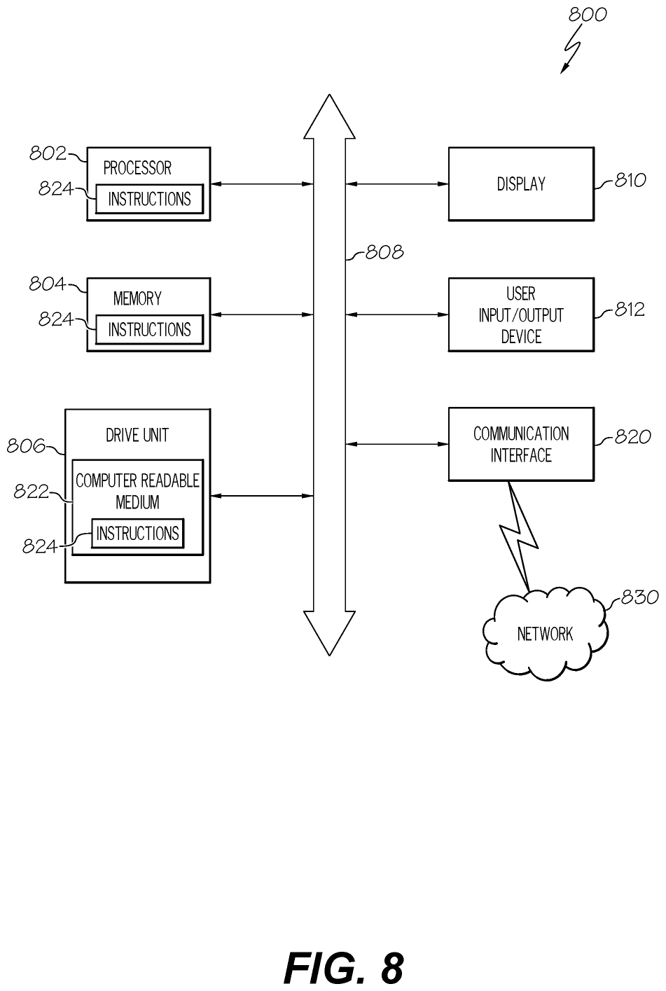

FIG. 8 depicts an implementation of a computer system that executes techniques presented herein, according to one or more embodiments. Computer system 800 can include a set of instructions that can be executed to cause the computer system 800 to perform any one or more of the methods or computer-based functions disclosed herein. The computer system 800 operates as a standalone device or is connected, e.g., using a network, to other computer systems or peripheral devices.

Unless specifically stated otherwise, as apparent from the following discussions, it is appreciated that throughout the specification, discussions utilizing terms such as “processing,” “computing,” “calculating,” “determining”, analyzing” or the like, refer to the action and/or processes of a computer or computing system, or similar electronic computing device, that manipulate and/or transform data represented as physical, such as electronic, quantities into other data similarly represented as physical quantities.

In a similar manner, the term “processor” refers to any device or portion of a device that processes electronic data, e.g., from registers and/or memory to transform that electronic data into other electronic data that, e.g., is stored in registers and/or memory. A “computer,” a “computing machine,” a “computing platform,” a “computing device,” or a “server” includes one or more processors.

In a networked deployment, the computer system 800 operates in the capacity of a server or as a client user computer in a server-client user network environment, or as a peer computer system in a peer-to-peer (or distributed) network environment. The computer system 800 can also be implemented as or incorporated into various devices, such as a personal computer (PC), a tablet PC, a set-top box (STB), a personal digital assistant (PDA), a mobile device, a palmtop computer, a laptop computer, a desktop computer, a communications device, a wireless telephone, a land-line telephone, a control system, a camera, a scanner, a facsimile machine, a printer, a pager, a personal trusted device, a web appliance, a network router, switch or bridge, or any other machine capable of executing a set of instructions (sequential or otherwise) that specify actions to be taken by that machine. In a particular implementation, the computer system 800 can be implemented using electronic devices that provide voice, video, or data communication. Further, while the computer system 800 is illustrated as a single system, the term “system” shall also be taken to include any collection of systems or sub-systems that individually or jointly execute a set, or multiple sets, of instructions to perform one or more computer functions.

As illustrated in FIG. 8, the computer system 800 includes a processor 802, e.g., a central processing unit (CPU), a graphics processing unit (GPU), or both. The processor 802 can be a component in a variety of systems. For example, the processor 802 is part of a standard personal computer or a workstation. The processor 802 is one or more processors, digital signal processors, application specific integrated circuits, field programmable gate arrays, servers, networks, digital circuits, analog circuits, combinations thereof, or other now known or later developed devices for analyzing and processing data. The processor 802 implements a software program, such as code generated manually (e.g., programmed).

The computer system 800 includes a memory 804 that can communicate via a bus 808. The memory 804 is a main memory, a static memory, or a dynamic memory. The memory 804 includes, but is not limited to computer readable storage media such as various types of volatile and non-volatile storage media, including but not limited to random access memory, read-only memory, programmable read-only memory, electrically programmable read-only memory, electrically erasable read-only memory, flash memory, magnetic tape or disk, optical media, and the like. In one implementation, the memory 804 includes a cache or random-access memory for the processor 802. In alternative implementations, the memory 804 is separate from the processor 802, such as a cache memory of a processor, the system memory, or other memory. The memory 804 can be an external storage device or database for storing data. Examples include a hard drive, compact disc (“CD”), digital video disc (“DVD”), memory card, memory stick, floppy disc, universal serial bus (“USB”) memory device, or any other device operative to store data. The memory 804 is operable to store instructions executable by the processor 802. The functions, acts or tasks illustrated in the figures or described herein are performed by the processor 802 executing the instructions stored in the memory 804. The functions, acts or tasks are independent of the particular type of instructions set, storage media, processor or processing strategy and are performed by software, hardware, integrated circuits, firm-ware, micro-code and the like, operating alone or in combination. Likewise, processing strategies can include multiprocessing, multitasking, parallel processing, and the like.

As shown, the computer system 800 further included a display 810, such as a liquid crystal display (LCD), an organic light emitting diode (OLED), a flat panel display, a solid-state display, a cathode ray tube (CRT), a projector, a printer or other now known or later developed display device for outputting determined information. The display 810 acts as an interface for the user to see the functioning of the processor 802, or specifically as an interface with the software stored in the memory 804 or in a drive unit 806.

Additionally or alternatively, the computer system 800 includes an input/output device 812 configured to allow a user to interact with any of the components of the computer system 800. The input/output device 812 is a number pad, a keyboard, or a cursor control device, such as a mouse, or a joystick, touch screen display, remote control, or any other device operative to interact with the computer system 800.

The computer system 800 also or alternatively includes the drive unit 806 implemented as a disk or optical drive. The drive unit 806 includes a computer-readable medium 822 in which one or more sets of instructions 824, e.g., software, can be embedded. Further, the sets of instructions 824 embody one or more of the methods or logic as described herein. The instructions 824 reside completely or partially within the memory 804 and/or within the processor 802 during execution by the computer system 800. The memory 804 and the processor 802 can also include computer-readable media as discussed above.

In some systems, the computer-readable medium 822 includes the sets of instructions 824 or receives and executes the sets of instructions 824 responsive to a propagated signal so that a device connected to a network 830 can communicate voice, video, audio, images, or any other data over the network 830. Further, the sets of instructions 824 are transmitted or received over the network 830 via a communication port or interface 820, and/or using the bus 808. The communication port or interface 820 is a part of the processor 802 or is a separate component. The communication port or interface 820 is created in software or is a physical connection in hardware. The communication port or interface 820 are configured to connect with the network 830, external media, the display 810, or any other components in the computer system 800, or combinations thereof. The connection with the network 830 is a physical connection, such as a wired Ethernet connection or is established wirelessly as discussed below. Likewise, the additional connections with other components of the computer system 800 are physical connections or are established wirelessly. The network 830 is alternatively directly connected to the bus 808.

While the computer-readable medium 822 is shown to be a single medium, the term “computer-readable medium” includes a single medium or multiple media, such as a centralized or distributed database, and/or associated caches and servers that store one or more sets of instructions. The term “computer-readable medium” also includes any medium that is capable of storing, encoding, or carrying a set of instructions for execution by a processor or that cause a computer system to perform any one or more of the methods or operations disclosed herein. In some examples, the computer-readable medium 822 is non-transitory, and is tangible.

The computer-readable medium 822 can include a solid-state memory such as a memory card or other package that houses one or more non-volatile read-only memories. The computer-readable medium 822 can be a random-access memory or other volatile re-writable memory. Additionally or alternatively, the computer-readable medium 822 can include a magneto-optical or optical medium, such as a disk or tapes or other storage device to capture carrier wave signals such as a signal communicated over a transmission medium. A digital file attachment to an e-mail or other self-contained information archive or set of archives are considered a distribution medium that is a tangible storage medium. Accordingly, the disclosure is considered to include any one or more of a computer-readable medium or a distribution medium and other equivalents and successor media, in which data or instructions are storable.

In an alternative implementation, dedicated hardware implementations, such as application specific integrated circuits, programmable logic arrays and other hardware devices, can be constructed to implement one or more of the methods described herein. Applications that include the apparatus and systems of various implementations can broadly include a variety of electronic and computer systems. One or more implementations described herein implement functions using two or more specific interconnected hardware modules or devices with related control and data signals that can be communicated between and through the modules, or as portions of an application-specific integrated circuit. Accordingly, the present system encompasses software, firmware, and hardware implementations.

The computer system 800 is connected to the network 830. The network 830 defines one or more networks including wired or wireless networks, such as the network 104 described in FIG. 1. The wireless network can be a cellular telephone network, an 802.11, 802.18, 802.20, or WiMAX network. Further, such networks include a public network, such as the Internet, a private network, such as an intranet, or combinations thereof, and utilize a variety of networking protocols now available or later developed including, but not limited to TCP/IP based networking protocols. The network 830 can include wide area networks (WAN), such as the Internet, local area networks (LAN), campus area networks, metropolitan area networks, a direct connection such as through a Universal Serial Bus (USB) port, or any other networks that allow for data communication. The network 830 is configured to couple one computing device to another computing device to enable communication of data between the devices. The network 830 generally is enabled to employ any form of machine-readable media for communicating information from one device to another. The network 830 includes communication methods by which information travels between computing devices. The network 830 can be divided into sub-networks. The sub-networks allow access to all of the other components connected thereto or the sub-networks restrict access between the components. The network 830 can be regarded as a public or private network connection and can include, for example, a virtual private network or an encryption or other security mechanism employed over the public Internet, or the like.

It will be understood that the steps of methods discussed are performed in one embodiment by an appropriate processor (or processors) of a processing (e.g., computer) system executing instructions (computer-readable code) stored in storage. It will also be understood that the disclosure is not limited to any particular implementation or programming technique and that the disclosure is implementable using any appropriate techniques for implementing the functionality described herein. The disclosure is not limited to any particular programming language or operating system.

It should be appreciated that in the above description of example embodiments of the present disclosure, various features of the present disclosure are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed embodiment requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this disclosure.

Furthermore, while some embodiments described herein include some but not other features included in other embodiments, combinations of features of different embodiments are meant to be within the scope of the disclosure, and form different embodiments, as would be understood by those skilled in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the embodiments.

In the description provided herein, numerous specific details are set forth. However, it is understood that embodiments of the present disclosure can be practiced without these specific details. In other instances, well-known methods, structures, and techniques have not been shown in detail in order not to obscure an understanding of this description.

Thus, while there has been described what are believed to be the preferred embodiments of the present disclosure, those skilled in the art will recognize that other and further modifications can be made thereto without departing from the spirit of the embodiments, and it is intended to claim all such changes and modifications as falling within the scope of the embodiments. For example, any formulas given above are merely representative of procedures that can be used. Functionality can be added or deleted from the block diagrams and operations are interchangeable among functional blocks. Steps can be added or deleted to methods described within the scope of the present disclosure.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations and implementations are possible within the scope of the disclosure. Accordingly, the disclosure is not to be restricted except in light of the attached claims and their equivalents.

The present disclosure further relates to the following aspects.

Example 1. A method performed by one or more processors of a computing system, the method comprising: receiving an entity attribute associated with an entity; generating an entity consideration dataset using the received entity attribute; providing the generated entity consideration dataset to a plurality of resource systems; receiving one or more responses from the plurality of resource systems in response to the provided entity consideration dataset; generating a resource consideration dataset using the received one or more responses; and matching a resource system of the plurality of resource systems with the entity based on the resource consideration dataset.

Example 2. The method of example 1, wherein generating the entity consideration dataset using the received entity attribute includes: receiving metadata regarding the entity attribute; extracting a feature from the received metadata, the extracted feature corresponding to a feature of a trained machine-learning based model for determining the entity consideration dataset for the entity attribute based on a learned association between the extracted feature and a resource system; and automatically determining, using the trained machine-learning based model, the entity consideration dataset for the entity attribute based on the extracted feature and the learned association between the extracted feature and the resource system, wherein the trained machine-learning based model was trained based at least in part on a first feature extracted from metadata regarding a patient attribute and a second feature extracted from metadata regarding a resource system related to the entity attribute.

Example 3. The method of example 2, wherein the trained machine-learning based model was trained by operations including: receiving first metadata regarding a first entity attribute; extracting a first feature from the received first metadata; receiving second metadata regarding a first resource system related to the first entity attribute; extracting a second feature from the received second metadata; training the machine-learning based model to learn an association between the first entity attribute and the first resource system related to the first entity attribute, based on the extracted first feature and the extracted second feature; and automatically determining, using the trained machine-learning based model, a first entity consideration set for the first entity attribute based on the extracted first feature and the learned association between the first entity attribute and the first resource system related to the first entity attribute.

Example 4. The method of any of the previous examples, wherein generating the resource consideration dataset using the received one or more responses includes: receiving metadata regarding a resource system associated with the one or more responses; extracting a feature from the received metadata, the extracted feature corresponding to a feature of a trained machine-learning based model for determining the resource consideration dataset for the resource system based on a learned association between the extracted feature and an entity outcome; and automatically determining, using the trained machine-learning based model, the resource consideration dataset for the resource system based on the extracted feature and the learned association between the extracted feature and the entity outcome, wherein the trained machine-learning based model was trained based at least in part on a first feature extracted from metadata regarding a resource system and a second feature extracted from metadata regarding an entity outcome related to the resource system.

Example 5. The method of example 4, wherein the trained machine-learning based model was trained by operations including: receiving first metadata regarding a first resource system; extracting a first feature from the received first metadata; receiving second metadata regarding a first entity outcome related to the first resource system; extracting a second feature from the received second metadata; training the machine-learning based model to learn an association between the first resource system and the first entity outcome related to the first resource system, based on the extracted first feature and the extracted second feature; and automatically determining, using the trained machine-learning based model, a first resource consideration dataset for the first resource system based on the extracted first feature and the learned association between the first resource system and the first entity outcome related to the first resource system.

Example 6. The method of any of the previous examples, wherein matching the resource system with the entity based on the resource consideration dataset includes: receiving an acceptance of a response, among the one or more responses, from the resource system, the entity, a related entity, or an authorized resource system for the entity, and providing a notification to the resource system of the acceptance of the response.

Example 7. The method of any of the previous examples, wherein generating the resource consideration dataset using the received one or more responses includes: generating a score for the resource consideration dataset; and generating a ranked list of resource consideration datasets, including the resource consideration dataset, based on the score for each of a plurality of resource consideration datasets.

Example 8. The method of example 7, wherein generating the ranked list of resource consideration datasets includes using a trained machine-learning based model.

Example 9. The method of example 7, wherein matching the resource system with the entity based on the resource consideration dataset includes: automatically accepting a response, among the one or more responses, from the resource system, among the plurality of resource systems, having a highest score in the ranked list of resource consideration datasets including the resource consideration dataset; and providing a notification to the resource system of the acceptance of the response.

Example 10. The method of any of the previous examples, wherein the entity attribute includes one or more of: a characteristic of the entity, a condition of the entity, or a treatment of the entity.

Example 11. The method of any of the previous examples, wherein the entity consideration dataset includes one or more of: a cost of providing a service to the entity, a likelihood of the entity needing additional services the resource system can provide, a likelihood of the entity needing additional services the resource system cannot provide, a probability of the entity having a condition that is not documented, or a demand and capacity planning of the resource system.

Example 12. The method of any of the previous examples, wherein the resource consideration dataset includes factors including one or more of: a community fit for the entity, a causal inference model forecasting an additional life expectancy by a service provided for each response, or an ability for the entity to meet a cost of services over a given time period.

Example 13. The method of example 12, wherein generating the resource consideration dataset using the received one or more responses includes: generating a score for the resource consideration dataset based on a weighting scheme for the factors; and generating a ranked list of resource consideration datasets, including the resource consideration dataset, based on the score for each of a plurality of resource consideration datasets.

Example 14. A system comprising: one or more processors; and at least one memory storing instructions that, when executed by the one or more processors, cause the one or more processors to perform operations including: receiving an entity attribute associated with an entity; generating an entity consideration dataset using the received entity attribute; providing the generated entity consideration dataset to a plurality of resource systems; receiving one or more responses from the plurality of resource systems in response to the provided entity consideration dataset; generating a resource consideration dataset using the received one or more responses; and matching a resource system of the plurality of resource systems with the entity based on the resource consideration dataset.

Example 15. The system of example 14, wherein one or more of generating the entity consideration dataset using the received entity attribute or generating the resource consideration dataset using the received one or more responses includes using a trained machine-learning based model.

Example 16. The system of any of examples 14-15, wherein the operations further include: receiving an acceptance of a response, among the one or more responses, from the resource system, the entity, a related entity, or an authorized resource system for the entity, and providing a notification to the resource system of the acceptance of the response.

Example 17. The system of any of examples 14-16, wherein generating the resource consideration dataset using the received one or more responses includes: generating a score for the resource consideration dataset; and generating a ranked list of resource consideration datasets, including the resource consideration dataset, based on the score for each of a plurality of resource consideration datasets.

Example 18. The system of example 17, wherein generating the ranked list of resource consideration datasets includes using a trained machine-learning based model.

Example 19. The system of any of examples 17-18, wherein matching the resource system with the entity based on the resource consideration dataset includes: automatically accepting a response, among the one or more responses, from the resource system, among the plurality of resource systems, having a highest score in the ranked list of resource consideration datasets including the resource consideration dataset; and providing a notification to the resource system of the acceptance of the response.

Example 20. A non-transitory computer readable medium storing instructions that, when executed by one or more processors, cause the one or more processors to perform operations comprising: receiving an entity attribute associated with an entity; generating an entity consideration dataset using the received entity attribute; providing the generated entity consideration dataset to a plurality of resource systems; receiving one or more responses from the plurality of resource systems in response to the provided entity consideration dataset; generating a resource consideration dataset using the received one or more responses; and matching a resource system of the plurality of resource systems with the entity based on the resource consideration dataset.

Other embodiments of the disclosure will be apparent to those skilled in the art from consideration of the specification and practice of the embodiments disclosed herein. It is intended that the specification and examples be considered as exemplary only, with a true scope and spirit of the embodiments being indicated by the following claims.

What is claimed is:

1. A method performed by one or more processors of a computing system, the method comprising:
receiving an entity attribute associated with an entity;
generating an entity consideration dataset using the received entity attribute;
providing the generated entity consideration dataset to a plurality of resource systems;
receiving one or more responses from the plurality of resource systems in response to the provided entity consideration dataset;
generating a resource consideration dataset using the received one or more responses; and
matching a resource system of the plurality of resource systems with the entity based on the resource consideration dataset.

2. The method of claim 1, wherein generating the entity consideration dataset using the received entity attribute includes:
receiving metadata regarding the entity attribute;
extracting a feature from the received metadata, the extracted feature corresponding to a feature of a trained machine-learning based model for determining the entity consideration dataset for the entity attribute based on a learned association between the extracted feature and a resource system; and
automatically determining, using the trained machine-learning based model, the entity consideration dataset for the entity attribute based on the extracted feature and the learned association between the extracted feature and the resource system, wherein the trained machine-learning based model was trained based at least in part on a first feature extracted from metadata regarding a patient attribute and a second feature extracted from metadata regarding a resource system related to the entity attribute.

3. The method of claim 2, wherein the trained machine-learning based model was trained by operations including:
receiving first metadata regarding a first entity attribute;
extracting a first feature from the received first metadata;
receiving second metadata regarding a first resource system related to the first entity attribute;
extracting a second feature from the received second metadata;
training the machine-learning based model to learn an association between the first entity attribute and the first resource system related to the first entity attribute, based on the extracted first feature and the extracted second feature; and
automatically determining, using the trained machine-learning based model, a first entity consideration set for the first entity attribute based on the extracted first feature and the learned association between the first entity attribute and the first resource system related to the first entity attribute.

4. The method of claim 1, wherein generating the resource consideration dataset using the received one or more responses includes:
receiving metadata regarding a resource system associated with the one or more responses;
extracting a feature from the received metadata, the extracted feature corresponding to a feature of a trained machine-learning based model for determining the resource consideration dataset for the resource system based on a learned association between the extracted feature and an entity outcome; and
automatically determining, using the trained machine-learning based model, the resource consideration dataset for the resource system based on the extracted feature and the learned association between the extracted feature and the entity outcome, wherein the trained machine-learning based model was trained based at least in part on a first feature extracted from metadata regarding a resource system and a second feature extracted from metadata regarding an entity outcome related to the resource system.

5. The method of claim 4, wherein the trained machine-learning based model was trained by operations including:
receiving first metadata regarding a first resource system;
extracting a first feature from the received first metadata;
receiving second metadata regarding a first entity outcome related to the first resource system;
extracting a second feature from the received second metadata;
training the machine-learning based model to learn an association between the first resource system and the first entity outcome related to the first resource system, based on the extracted first feature and the extracted second feature; and
automatically determining, using the trained machine-learning based model, a first resource consideration dataset for the first resource system based on the extracted first feature and the learned association between the first resource system and the first entity outcome related to the first resource system.

6. The method of claim 1, wherein matching the resource system with the entity based on the resource consideration dataset includes:
receiving an acceptance of a response, among the one or more responses, from the resource system, the entity, a related entity, or an authorized resource system for the entity, and
providing a notification to the resource system of the acceptance of the response.

7. The method of claim 1, wherein generating the resource consideration dataset using the received one or more responses includes:
generating a score for the resource consideration dataset; and
generating a ranked list of resource consideration datasets, including the resource consideration dataset, based on the score for each of a plurality of resource consideration datasets.

8. The method of claim 7, wherein generating the ranked list of resource consideration datasets includes using a trained machine-learning based model.

9. The method of claim 7, wherein matching the resource system with the entity based on the resource consideration dataset includes:
automatically accepting a response, among the one or more responses, from the resource system, among the plurality of resource systems, having a highest score in the ranked list of resource consideration datasets including the resource consideration dataset; and
providing a notification to the resource system of the acceptance of the response.

10. The method of claim 1, wherein the entity attribute includes one or more of: a characteristic of the entity, a condition of the entity, or a treatment of the entity.

11. The method of claim 1, wherein the entity consideration dataset includes one or more of: a cost of providing a service to the entity, a likelihood of the entity needing additional services the resource system can provide, a likelihood of the entity needing additional services the resource system cannot provide, a probability of the entity having a condition that is not documented, or a demand and capacity planning of the resource system.

12. The method of claim 1, wherein the resource consideration dataset includes factors including one or more of: a community fit for the entity, a causal inference model forecasting an additional life expectancy by a service provided for each response, or an ability for the entity to meet a cost of services over a given time period.

13. The method of claim 12, wherein generating the resource consideration dataset using the received one or more responses includes:
generating a score for the resource consideration dataset based on a weighting scheme for the factors; and
generating a ranked list of resource consideration datasets, including the resource consideration dataset, based on the score for each of a plurality of resource consideration datasets.

14. A system comprising:
one or more processors; and
at least one memory storing instructions that, when executed by the one or more processors, cause the one or more processors to perform operations including:
receiving an entity attribute associated with an entity;
generating an entity consideration dataset using the received entity attribute;
providing the generated entity consideration dataset to a plurality of resource systems;
receiving one or more responses from the plurality of resource systems in response to the provided entity consideration dataset;
generating a resource consideration dataset using the received one or more responses; and
matching a resource system of the plurality of resource systems with the entity based on the resource consideration dataset.

15. The system of claim 14, wherein one or more of generating the entity consideration dataset using the received entity attribute or generating the resource consideration dataset using the received one or more responses includes using a trained machine-learning based model.

16. The system of claim 14, wherein the operations further include:
receiving an acceptance of a response, among the one or more responses, from the resource system, the entity, a related entity, or an authorized resource system for the entity, and
providing a notification to the resource system of the acceptance of the response.

17. The system of claim 14, wherein generating the resource consideration dataset using the received one or more responses includes:
generating a score for the resource consideration dataset; and
generating a ranked list of resource consideration datasets, including the resource consideration dataset, based on the score for each of a plurality of resource consideration datasets.

18. The system of claim 17, wherein generating the ranked list of resource consideration datasets includes using a trained machine-learning based model.

19. The system of claim 17, wherein matching the resource system with the entity based on the resource consideration dataset includes:
automatically accepting a response, among the one or more responses, from the resource system, among the plurality of resource systems, having a highest score in the ranked list of resource consideration datasets including the resource consideration dataset; and
providing a notification to the resource system of the acceptance of the response.

20. A non-transitory computer readable medium storing instructions that, when executed by one or more processors, cause the one or more processors to perform operations comprising:
receiving an entity attribute associated with an entity;
generating an entity consideration dataset using the received entity attribute;
providing the generated entity consideration dataset to a plurality of resource systems;
receiving one or more responses from the plurality of resource systems in response to the provided entity consideration dataset;
generating a resource consideration dataset using the received one or more responses; and
matching a resource system of the plurality of resource systems with the entity based on the resource consideration dataset.
